(19) United States
(12) Patent Application Publication
(10) Pub. No.: US 2013/0296667 A1
(43) Pub. Date: Nov. 7, 2013

(54) Sensor Patch For Detecting and Alerting Of A Cardiac Abnormality

(71) Applicants: Jahangir S. Rastegar, Stony Brook, NY (US); Thomas Spinelli, Northport, NY (US)

(72) Inventors: Jahangir S. Rastegar, Stony Brook, NY (US); Thomas Spinelli, Northport, NY (US)

(73) Assignee: OMNITEK PARTNERS LLC, Ronkonkoma, NY (US)

(21) Appl. No.: 13/464,973

(22) Filed: May 5, 2012

(51) Int. Cl. A61B 5/02; A61B 8/00; A61B 5/1455; A61B 5/0456; A61B 5/0452

(52) U.S. Cl. 600/323; 600/508; 600/521; 600/515; 600/459

(57) ABSTRACT

A system for monitoring a cardiac characteristic. The system including: a sensor patch adhered to a surface of the skin which includes: a patch body having a surface and an adhesive, the patch body further having a cavity; a sensor disposed in the cavity and configured for detecting the cardiac characteristic; a transmitter connected to the sensor for transmitting a signal representative of the cardiac characteristic; and a power source disposed in the cavity for powering the sensor and transmitter; and a receiver for receiving or monitoring the signal from the transmitter and outputting information associated with the cardiac characteristic. The receiver can be a cellular phone having an application used to receive the signal from the transmitter and generate the information associated with the cardiac characteristic. The cardiac characteristic can be a cardiac abnormality or useful in monitoring or providing feedback to the user while exercising.

### BACKGROUND

1. Field

The present invention relates generally to cardiac sensors; and more particularly to a sensor patch for detecting and alerting of cardiac abnormalities.

2. Prior Art

Cardiac sensing devices are known in the art, such as those carried on a halter by a patient. Although such devices have utility they suffer from being cumbersome and bulky and the motions of everyday life interfere with their use, such as showering or other contact with water. Furthermore, motions of everyday like may act to disable their use, such as loosening a necessary contact with the skin or a contact with a wire and the contact or with a wire and the device body.

### SUMMARY

Accordingly, a sensor patch for adherence to a surface of the skin of a user is provided. The sensor patch comprising: a patch body having a surface and an adhesive disposed on the surface, the patch body further having a cavity; a sensor configured for detecting a characteristic useful in determining a cardiac abnormality disposed in the cavity; a transmitter operatively connected to the sensor for one of transmitting a signal or discontinuing transmittance of a signal upon an occurrence of the cardiac abnormality detected by the sensor; and a power source disposed in the cavity for powering the sensor and transmitter.

The sensor further comprises a release layer removably secured to the surface of the patch body to cover the adhesive.

The cavity can be sealed against moisture and other harmful contaminants.

The characteristic can be selected from a group consisting of R-waves, heart rate, blood oxygen and arrhythmia.

The sensor can be configured to operate with one or more conductive contacts to detect the characteristic. The one or more conductive contacts can comprise the adhesive, which is a conductive adhesive.

The sensor can be an ultrasound sensor which uses ultrasound to detect the characteristic. The adhesive can have ultrasound signal coupling characteristics corresponding to the ultrasound sensor.

The transmitter can comprise a transceiver.

The sensor patch can further comprise a memory for storing data and a processor disposed in the cavity for controlling one or more of the sensor, transmitter and memory.

Also provided is a system for monitoring for a cardiac abnormality in a user. The system comprising: a sensor patch for adherence to a surface of the skin of the user, the sensor patch comprising: a patch body having a surface and an adhesive disposed on the surface, the patch body further having a cavity; a sensor configured for detecting a characteristic useful in determining a cardiac abnormality disposed in the cavity; a transmitter operatively connected to the sensor for one of transmitting a signal or discontinuing transmittance of a signal upon an occurrence of the cardiac abnormality detected by the sensor; and a power source disposed in the cavity for powering the sensor and transmitter; and a receiver for receiving or monitoring the signal from the transmitter and outputting information associated with the occurrence of the cardiac abnormality.

The receiver can be a cellular phone having an application used to receive the signal from the transmitter and generate the information associated with the occurrence of the cardiac abnormality.

Still further provided is a method for monitoring for a cardiac abnormality in a user. The method comprising: adhering a sensor patch to a surface of the skin of the user; detecting a characteristic useful in determining a cardiac abnormality using a sensor disposed in a cavity of the sensor patch; and one of transmitting a signal or discontinuing transmittance of a signal from the sensor patch upon an occurrence of the cardiac abnormality detected by the sensor.

The can further comprise receiving or monitoring the signal from the transmitter and outputting information associated with the occurrence of the cardiac abnormality. The receiver can be a cellular phone having an application used to receive the signal from the transmitter and generate the information associated with the occurrence of the cardiac abnormality.

The one of transmitting a signal or discontinuing transmittance of the signal from the sensor can be repeated a plurality of times. The repeating of the one of transmitting a signal or discontinuing transmittance of the signal from the sensor can continue until the sensor patch receives a signal indicating that the transmitted signal has been received by a receiver.

The information can be selected from a group consisting of contacting emergency services, providing a canned message to emergency services, contacting one or predetermined persons of the cardiac abnormality, providing information of the type of cardiac abnormality detected, providing stored information about the user, providing location information to emergency services and providing instructions on how or where to treat the user for the cardiac abnormality.

The information can include location information, where the location information can be generated by one or more of a GPS receiver disposed in the receiver or based on triangulation of signals received by the receiver.

The occurrence of the cardiac abnormality can be determined by comparing the detected characteristic to a threshold value stored in a memory.

Still further yet provided is a system for monitoring a cardiac characteristic of a user. The system comprising: a sensor patch for adherence to a surface of the skin of the user, the sensor patch comprising: a patch body having a surface and an adhesive disposed on the surface, the patch body further having a cavity; a sensor disposed in the cavity and configured for detecting the cardiac characteristic; a transmitter operatively connected to the sensor for transmitting a signal representative of the cardiac characteristic detected by the sensor; and a power source disposed in the cavity for powering the sensor and transmitter; and a receiver for receiving or monitoring the signal from the transmitter and outputting information associated with the cardiac characteristic.

The receiver can be a cellular phone having an application used to receive the signal from the transmitter and generate the information associated with the cardiac characteristic.

The cardiac characteristic can be a cardiac abnormality.

The cardiac characteristic can be useful in monitoring or providing feedback to the user while exercising.

### BRIEF DESCRIPTION OF THE DRAWINGS

These and other features, aspects, and advantages of the apparatus of the present invention will become better understood with regard to the following description, appended claims, and accompanying drawings where:

FIG. 1A illustrates a top schematic view of a sensor patch of a first embodiment, with a cover not shown so as to illustrate interior components.

FIG. 1B illustrates a side view of the sensor patch of FIG. 1A.

FIG. 2 illustrates a top schematic view of a sensor patch of a second embodiment.

FIG. 3 illustrates a sensor patch system for detecting and alerting of a cardiac abnormality.

### DETAILED DESCRIPTION

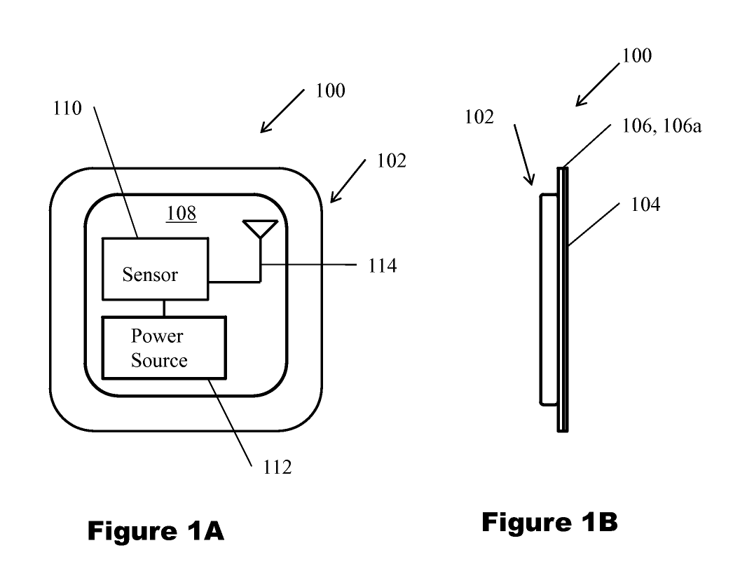

Referring now to FIGS. 1A and 1B, there is shown a sensor patch of a first embodiment, generally referred to by reference numeral 100. The sensor patch 100 includes a patch body 102 and a release layer 104 that is removable secured to a lower surface 106 of the patch body 102. The lower surface 106 of the patch body 102 includes an adhesive 106a for temporarily securing the patch body 102 to a surface of a patient's body, such as a surface of the chest. Such adhesives are well known in the art of bandages and dressings. The sensor patch 100 is shown to be rectangular in shape and configured to be adhered to a relatively flat surface, however, those skilled in the art will appreciate that the sensor patch 100 can be configured in any shape and size and for adherence to any shape or size surface. For example, the sensor patch 100 can be configured to be adhered to a curved surface, such as the neck or finger of the human body (hereinafter referred to as the user or patient). The components (discussed below) disposed in the body 102 are maintained is a sealed state so as to protect such components against moisture and other harmful environments. Those skilled in the art will appreciate many means for sealing components in a package, such as using sealing materials to construct the patch body 102, such as plastic and elastomer materials that are water resistant. Furthermore, the sensor patch 100 can be sealed against moisture and other harmful environments by providing a sealing membrane to surround the components in the package or encapsulating the components in the cavity (see 108 below) in a gel or an epoxy.

The patch body 102 includes a cavity 108 containing a sensor 110 configured to sense a characteristic useful in determining a cardiac abnormality. Such characteristics include those capable of detecting R-waves (i.e., EKG signal), heart rate, blood oxygen, arrhythmia and the like. The sensor 110 is powered by a power source 112, such as one or more cell batteries operatively connected to the sensor 110, such as through internal wiring or conductive contacts. The body 102 further has a transmitter 114 disposed therein or thereon for transmitting a signal from the sensor patch 100 to the outside for broadcast. The internal components disposed in the cavity 108 can also be mounted on an integral substrate, such as a flexible membrane, having integral wiring and/or contacts. Where the sensor operates together with conductive contacts, the adhesive 106a used to connect the sensor patch 100 to the patient's body can be electrically conductive to allow detection of such electrical signals, such as the EKG signals. Alternatively, contacts can be disposed on surface 106 and operatively connected to the sensor 110. Where the sensor 110 is an ultrasound type sensor, an adhesive with proper ultrasound signal coupling characteristics can be employed.

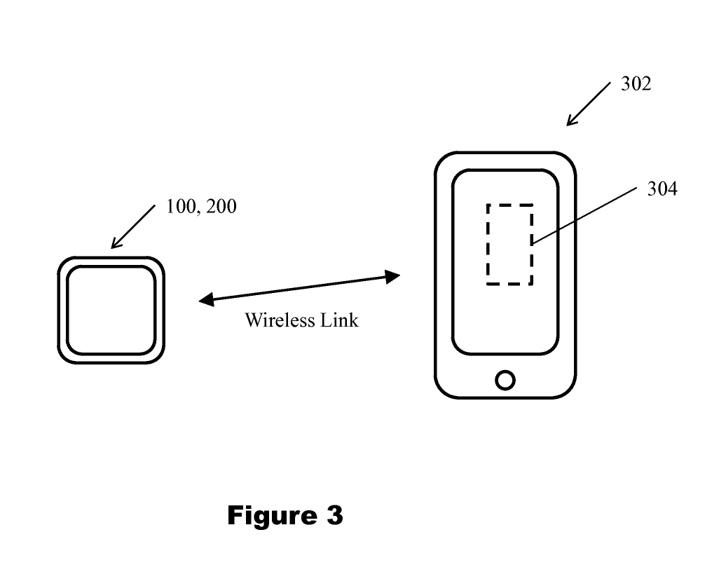

In a simplest configuration, when the sensor 110 detects a cardiac abnormality, such as an absence of heart rate, there is a resulting change in output at the transmitter 114, such as output of a signal or discontinuance of an output signal. For example, when the sensor 110 detects a cardiac abnormality, a signal, indicating the detected cardiac abnormality is transmitted from the transmitter 114. The transmitted signal can be transmitted multiple times. Furthermore, the transmitter 114 can be a transceiver, which could continue transmitting the aforementioned signal indicating the detected cardiac abnormality until it receives a signal indicating that the transmitted signal has been received by a receiving device, for example, the receiver 302 shown in FIG. 3.

Referring now to FIG. 3, the sensor patch 100 is used in connection with a receiver 302, such as a cell phone running an application 304 specific to the sensor patch 100. The receiver 302 is configured, through the application 304, to detect the transmission of the signal from the transmitter 114 and process the same. For example, the cell phone, under the control of the application 304, may call 911 emergency services, through its built in cellular network or Wi-Fi network and provide a canned message to a 911 operator alerting the operator of the cardiac abnormality and possibly to the family and/or friends and/or a close hospital and/or personal physician. The cell phone may also provide information of the type of abnormality detected as well as stored information about the user, such as name, address, person to contact in case of emergency, blood type, allergies to medicines, past medical history and the like. The cell phone may also transmit to the 911 operator, location information of the person wearing the patch and having the cell phone. The location information may be GPS data from a GPS receiver on-board the cell phone or location data based on triangulation of signals from nearby cell towers. The transmitted information may also include the location of the patient in the house and the location of a key for getting into the house, etc.

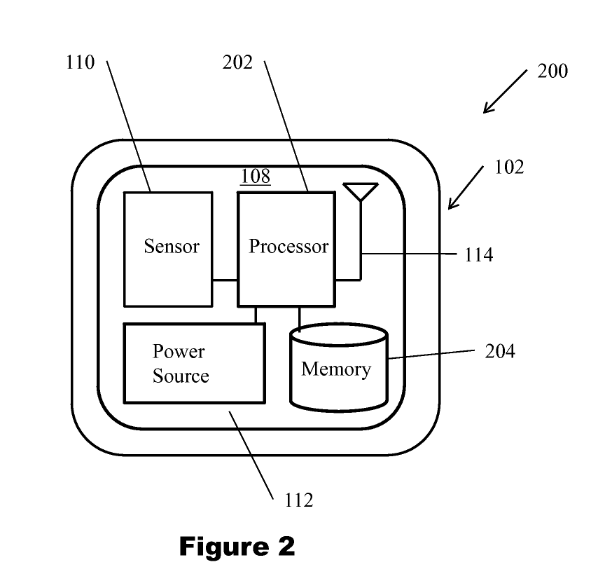

Referring now to FIG. 2, there is shown another embodiment of a sensor patch, generally referred to by reference numeral 200 and in which like reference numerals denote like features from FIGS. 1A and 1B. Like FIG. 1A, the cover of the sensor patch 200 is not shown for illustration of internal components. The sensor patch 200 can be used in the system illustrated in FIG. 3.

In addition to the components discussed with regard to FIG. 1A, the sensor patch 200 also includes a processor 202 and memory 204. The processor 202 allows more complicated monitoring, which can compare the detected characteristic to a threshold value stored in the memory 204 and the processor can decide whether to transmit the signal based on the comparison of the detected characteristic with the threshold value stored in the memory 204. The type of signal and/or information transmitted in the signal from the sensor patch 200 can be more complicated where the memory 204 and processor 202 are used.

Of course, the sensor patch may be configured in other ways known in the art, such as with an RF tag that transmits an RF signal to the receiver, such as the cell phone. Bluetooth standard may also be established between the sensor patch and the receiver. Further multiple sensor patches can be used with a single receiver, in which case, the sensor patches can detect the same or different cardiac characteristics and the multiple sensor patches may be associated with the same user or multiple users. In the case of the latter, the application running on the receiver, can track the different users and have data stored therein associated with each of the multiple users. Similarly, each sensor patch may include more than one sensor, for either or both of providing a duplicate of the same sensor and/or for providing detection capabilities for more than one characteristic. Although described in regard to a mobile receiver, such as a cell phone, the receiver can also be associated with a location, such as a desktop computer having a software or internet application. The receiver can also be a device dedicated to operate with the sensor patch (or multiple sensor patches).

In another embodiment of the sensor patch 100, 200 discussed above, the same may be configured to interface with an exercise and/or dieting application running on the receiver 302 (i.e., cell phone). In such a configuration, the sensor 110 can monitor the heart rate or other cardiac characteristic (such as blood oxygen) and the application 304 can use the cardiac characteristic, for example, to determine if the user is exercising so as to burn fat or build stamina when used with an exercise application, or the number of calories being burned when used with a diet application. The receiver 302 can provide feedback, for example, to optimize the exercise for its intended use (e.g., to burn calories). Also, the receiver can double as described above, that is, the receiver 302 can monitor for a cardiac abnormality while the use exercises.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A sensor patch for adherence to a surface of the skin of a user, the sensor patch comprising:
a patch body having a surface and an adhesive disposed on the surface, the patch body further having a cavity;
a sensor configured for detecting a characteristic useful in determining a cardiac abnormality disposed in the cavity;
a transmitter operatively connected to the sensor for one of transmitting a signal or discontinuing transmittance of a signal upon an occurrence of the cardiac abnormality detected by the sensor; and
a power source disposed in the cavity for powering the sensor and transmitter.

2. The sensor patch of claim 1, further comprising a release layer removably secured to the surface of the patch body to cover the adhesive.

3. The sensor patch of claim 1, wherein the cavity sealed against moisture and other harmful contaminants.

4. The sensor patch of claim 1, wherein the characteristic is selected from a group consisting of R-waves, heart rate, blood oxygen and arrhythmia.

5. The sensor patch of claim 1, wherein the sensor is configured to operate with one or more conductive contacts to detect the characteristic.

6. The sensor patch of claim 5, wherein the one or more conductive contacts comprise the adhesive, which is a conductive adhesive.

7. The sensor patch of claim 1, wherein the sensor is an ultrasound sensor which uses ultrasound to detect the characteristic.

8. The sensor patch of claim 7, wherein the adhesive has ultrasound signal coupling characteristics corresponding to the ultrasound sensor.

9. The sensor patch of claim 1, wherein the transmitter comprises a transceiver.

10. The sensor patch of claim 1, further comprising a memory for storing data and a processor disposed in the cavity for controlling one or more of the sensor, transmitter and memory.

11. A system for monitoring for a cardiac abnormality in a user, the system comprising:
a sensor patch for adherence to a surface of the skin of the user, the sensor patch comprising:
a patch body having a surface and an adhesive disposed on the surface, the patch body further having a cavity;
a sensor configured for detecting a characteristic useful in determining a cardiac abnormality disposed in the cavity;
a transmitter operatively connected to the sensor for one of transmitting a signal or discontinuing transmittance of a signal upon an occurrence of the cardiac abnormality detected by the sensor; and
a power source disposed in the cavity for powering the sensor and transmitter; and
a receiver for receiving or monitoring the signal from the transmitter and outputting information associated with the occurrence of the cardiac abnormality.

12. The system of claim 11, wherein the receiver is a cellular phone having an application used to receive the signal from the transmitter and generate the information associated with the occurrence of the cardiac abnormality.

13. A method for monitoring for a cardiac abnormality in a user, the method comprising:
adhering a sensor patch to a surface of the skin of the user;
detecting a characteristic useful in determining a cardiac abnormality using a sensor disposed in a cavity of the sensor patch; and
one of transmitting a signal or discontinuing transmittance of a signal from the sensor patch upon an occurrence of the cardiac abnormality detected by the sensor.

14. The method of claim 13, further comprising receiving or monitoring the signal from the transmitter and outputting information associated with the occurrence of the cardiac abnormality.

15. The method of claim 14, wherein the receiver is a cellular phone having an application used to receive the signal from the transmitter and generate the information associated with the occurrence of the cardiac abnormality.

16. The method of claim 13, wherein the one of transmitting a signal or discontinuing transmittance of the signal from the sensor is repeated a plurality of times.

17. The method of claim 16, wherein the repeating of the one of transmitting a signal or discontinuing transmittance of the signal from the sensor continues until the sensor patch receives a signal indicating that the transmitted signal has been received by a receiver.

18. The method of claim 13, wherein the information is selected from a group consisting of contacting emergency services, providing a canned message to emergency services, contacting one or predetermined persons of the cardiac abnormality, providing information of the type of cardiac abnormality detected, providing stored information about the user, providing location information to emergency services and providing instructions on how or where to treat the user for the cardiac abnormality.

19. The method of claim 13, wherein when the information includes location information, the location information can be generated by one or more of a GPS receiver disposed in the receiver or based on triangulation of signals received by the receiver.

20. The method of claim 13, wherein the occurrence of the cardiac abnormality is determined by comparing the detected characteristic to a threshold value stored in a memory.

21. A system for monitoring a cardiac characteristic of a user, the system comprising:
a sensor patch for adherence to a surface of the skin of the user, the sensor patch comprising:
a patch body having a surface and an adhesive disposed on the surface, the patch body further having a cavity;
a sensor disposed in the cavity and configured for detecting the cardiac characteristic;
a transmitter operatively connected to the sensor for transmitting a signal representative of the cardiac characteristic detected by the sensor; and
a power source disposed in the cavity for powering the sensor and transmitter; and
a receiver for receiving or monitoring the signal from the transmitter and outputting information associated with the cardiac characteristic.

22. The system of claim 21, wherein the receiver is a cellular phone having an application used to receive the signal from the transmitter and generate the information associated with the cardiac characteristic.

23. The system of claim 22, wherein the cardiac characteristic is a cardiac abnormality.

24. The system of claim 22, wherein the cardiac characteristic is useful in monitoring or providing feedback to the user while exercising.
